(19) United States
(12) Patent Application Publication
(10) Pub. No.: US 2006/0024277 A1
(43) Pub. Date: Feb. 2, 2006

(54) Method of skin care and/or treatment using extracts enriched in mitochondria

(76) Inventors: Hannah Naomi Sivak, Gilbert, AZ (US); Alberto Alvaro Iglesias, Rosario (AR); Miguel Angel Ballicora, Skokie, IL (US)

Correspondence Address: Hannah Naomi Sivak, 1213 W Seascape Dr., Gilbert, AZ, 85233, US

(21) Appl. No.: 11/190,606

(22) Filed: Jul. 27, 2005

Related U.S. Application Data
(60) Provisional application No. 60/591,341, filed on Jul. 27, 2004.

(51) Int. Cl. A61K 38/44; A61K 38/05; A61K 31/375; A61K 31/685; A61K 31/355; A61K 31/195

(52) U.S. Cl. 424/93.7; 514/18; 514/78; 514/458; 514/474; 424/94.4; 514/563

(57) ABSTRACT

Methods for the prevention and treatment of skin aging arising from depletion of components of the mitochondrial membranes or the respiratory system using extracts rich in mitochondrial components in a dermatologically acceptable carrier that can be topically applied to the skin areas, including lips. In some embodiments, proteins such as epidermal growth factor and thioredoxin, or antioxidants such as reduced glutathione and astaxanthin, or lipids such as cardiolipin, are used.

### RELATED APPLICATION DATA

This application claims the benefit of U.S. Provisional Application No. 60/591,341, filed on Jul. 27, 2004.

### BACKGROUND OF THE INVENTION

Mitochondria are the powerhouses of the cell: they convert chemical energy stored as sugars, amino acids, fatty acids, etc. into ATP that the cell can use to maintain its structure and to grow and reproduce.

Unfortunately, mitochondrial activity, which is in essence organized oxidation, exposes the organelle to the presence of damaging oxygen species which are a side product of oxidation via the electron transport chain.

Mitochondria contain enzymes capable of protecting against the deleterious effects of free radicals, like superoxide dismutase in the case of superoxide. These protective mechanisms, however, are not perfect, and free radicals damage mitochondrial membranes, whose integrity is essential for optimal activity. Other effects of reactive oxygen species include a relatively high rate of mitochondrial DNA mutation.

Mitochondrial aging seems to occur at a faster pace than that of other cellular organelles, a problem that has been attributed to the high DNA mutation rate. Mitochondrial changes associated with aging are many and varied. They include, for example, a change in the flexibility of the mitochondrial membranes, and decreases in the content of omega-3 poly-unsaturated fatty acids and cardiolipin, decrease in the activity of many enzymes and changes in the degree of coupling of electron transport (Paradies et. al. 1996).

The effect of premature aging of mitochondria on the whole organism has been shown to be devastating and far reaching (Kujoth et al., 2005). Although those studies were concerned with a defective DNA polymerase causing premature mitochondrial aging, it is to be expected that damage to mitochondria due to any factor, e.g. free radicals, would be just as damaging to the whole organism.

There is also abundant scientific research suggesting an important role of mitochondria in the health of animal tissues. Some examples:
1) Mitochondrial dysfunction, due to either environmental or genetic factors, can result in excessive production of reactive oxygen species, triggering the apoptotic death of dopaminergic cells in Parkinson's disease (Fiskum et al, 2003)
2) Mitochondrial abnormalities seem to be involved in the degenerative process of Alzheimer's disease
3) A mitochondrial defect (low ubiquinol-cytochrome c reductase orcomplex III) in the respiratory chain causes miopathy. Darley-Usmar, V. et al., 1986)
4) Batten Disease, a group of neurodegenerative diseases, is apparently caused by a mitochondrial defect (Tanner, A. et al., 1996).

### DETAILED DESCRIPTION OF THE INVENTION

The composition and function of plant and yeast mitochondria are not that different from those of mammals.

Although the role of skin is to provide a permeability barrier between the body and the outside environment, the skin is not a perfect barrier and the barrier integrity decreases with age (Fore-Pfliger J., 2004a,b). Components of the plant or yeast mitochondrial extracts applied topically to mammalian skin will be absorbed and help replenish depleted or damaged mitochondrial components of aging skin cells.

Several methods to isolate intact mitochondria from yeast or plants have been described in previous art. The usual methods involve the isolation of intact mitochondria from crude extracts or from isolated protoplasts. It is important to obtain intact mitochondria because the final extract will then contain both soluble and membrane-associated components, while broken mitochondria would be mostly devoid of the original soluble components.

1) Preparation of mitochondria from crude extracts. Using a suitable plant material, e.g. mung beans hypocotyls or cauliflower florets, the tissue is disrupted in a suitable isotonic media. Mitochondria are separated from the other cell components by differential centrifugation (Bowman et al. 1976).

2) Preparation of mitochondria from protoplasts. To preserve the integrity of the mitochondria, (thus avoiding loss of mitochondrial contents) cell walls are digested using cellulase, hemicellulase and/or pectinase. The purified protoplasts are then broken using minimal force, and intact mitochondria are then separated by differential centrifugation as described above (Nishimura et. al. 1982).

Once separated, intact mitochondria obtained by any method are broken before adding to the carrier that will facilitate topical application of the extract. Mitochondria can be broken by repeated freezing and thawing or by exposing them to a suitable hypotonic medium.

While the carrier for the mitochondria extract can be very simple (such as saline solution), it is generally preferred that the carrier be a composition that will facilitate topical application, and particularly one which will favor absorption of its components and form a film or layer on the skin to which it is applied so as to localize the active ingredient. Many such compositions are known in the art, and can take the form of lotions, creams, gels, etc. Typical compositions include lotions containing water and/or alcohols and emollients such as natural oils and waxes, silicone oils, hyaluronic acid, glyceride derivatives, fatty acids or fatty acid esters or alcohols or alcohol ethers, lanolin and derivatives, polyhydric alcohols or esters, wax esters, sterols, phospholipids and the like, and generally also emulsifiers (nonionic, cationic or anionic), although some of the emollients inherently possess emulsifying properties. These same general ingredients can be formulated into a cream rather than a lotion, or into gels, or into solid sticks by utilization of different proportions of the ingredients and/or by inclusion of thickening agents such as gums or other forms of hydrophilic colloids. Such compositions are referred to herein as dermatologically-acceptable carriers.

Many preferred embodiments of this invention contain at least one or two, and sometimes several, other active ingredients in addition to mitochondria extract, provided that the ingredients are not acids present in concentrations high enough to denature and the mitochondria extract components sensitive to low pH.

Some embodiments may include materials to maintain components of the mitochondrial electron transport chain in its reduced state.

Antioxidants such as tocotrienol, lycopene, astaxanthin, ascorbic acid, and/or vitamin E may also be added to the mitochondria extract composition, alone or in combination with reduced glutathione in some embodiments.

In terms of a possible explanation for the effectiveness of the active ingredients in the prevention or treatment of damage to the skin, it is noted that some or all components of the plant mitochondrial extract will replenish skin mitochondrial components depleted or damaged. Just like in the living cell, where a number of components work in a concerted fashion, some embodiments of this invention also use the synergistic effect of antioxidants or other mitochondrial components such as reduced glutathione, tocotrienols, vitamin E, ascorbic acid, superoxide dismutase, catalase, astaxanthin, lycopene, epidermal growth factor, cardiolipin, superoxide dismutase.

The method of the present invention is particularly useful for the prevention and treatment of sunburn and other skin damage resulting from exposure to ultraviolet radiation, which accelerates skin aging. Mitochondria extract, alone or with other active ingredients can thus be added to dermatological creams and emollients as well as to commercial sunscreens to enhance their sun protection activity, or to creams used to treat sunburn or burns produced by therapeutical radiation used to treat cancer.

Having described the invention with reference to particular compositions, theories of effectiveness, it will be apparent to those of skill in the art that it is not intended that the invention be limited by such illustrative embodiments or mechanisms, and that modifications can be made without departing from the scope or spirit of the invention, as defined by the appended claims. It is intended that all modifications and variations be included within the scope of the invention. The claims are meant to cover the claimed components and steps in any sequence which is effective to meet the objectives there intended, unless the context specifically indicates the contrary.

### RELEVANT REFERENCES

Bowman, E. J. et al. Citric Acid Cycle Activity in Mitochondria Isolated from Mung Bean Hypocotyls. Plant Physiol. (1976) 58: 426-432.
Darley-Usmar, V. et al. Mitochondrial myopathy: tissue-specific expression of a defect in ubiquinol-cytochrome c reductase. Clinica Chimica Acta (1986), 158(3), 253-61.
Fiskum, G. et al. Mitochondrial mechanisms of neural cell death and neuroprotective interventions in Parkinson's disease. Annals of the New York Academy of Sciences (2003), 991: 111-119.
The epidermal skin barrier: implications for the wound care practitioner, part II. Fore-Pfliger J. Advances in skin & wound care (2004), 17:480-488
The epidermal skin barrier: implications for the wound care practitioner, part I. Fore-Pfliger J. Advances in skin & wound care (2004) 17:417-425.
Kujoth, G. C. et al. Mitochondrial DNA Mutations, Oxidative Stress, and Apoptosis in Mammalian Aging. Science (Washington, D.C., United States) (2005), 309: 481-484.
Nishimura, M. et al. Isolation and Characterization of Metabolically Competent Mitochondria from Spinach Leaf Protoplasts, Plant Physiol. (1982) 69: 916-920.
Paradies, G. et al. Age-dependent impairment of mitochondrial function in rat heart tissue. Effect of pharmacological agents. Annals of the New York Academy of Sciences (1996), 786: 252-263.
Swerdlow, R. and Kish, S. J. Mitochondria in Alzheimer's disease. International Review of Neurobiology (2002), 53: 341-385.
Tanner, A. et al. Batten disease and mitochondrial pathways of proteolysis. Biochemical and Molecular Medicine (1996), 57(1), 1-9.

We claim:

1. A method for the treatment and prevention of skin damage brought about by natural aging, or exposure to sun and other types of radiation or stressors, which consists of applying a composition containing extracts enriched in mitochondrial components in a dermatologically acceptable carrier to the affected skin area.

2. A method in accordance with claim 1 herein said composition further comprises one or more additional ingredients selected from the group consisting of reduced glutathione, tocotrienols, vitamin E, ascorbic acid, superoxide dismutase, catalase, astaxanthin, lycopene, epidermal growth factor, cardiolipin, superoxide dismutase.

3. A method in accordance with claims 1 or 2, wherein said composition is used to ameliorate skin conditions or diseases related to or leading to mitochondrial dysfunction.
